(19) United States
(12) Patent Application Publication
(10) Pub. No.: US 2020/0022602 A1
(43) Pub. Date: Jan. 23, 2020

(54) VEHICULAR ELECTROCARDIOGRAM MEASUREMENT DEVICE AND METHOD

(71) Applicant: LG Electronics Inc., Seoul (KR)

(72) Inventors: Nam Yong PARK, Gyeonggi-do (KR); Kwang Hee LEE, Gyeonggi-do (KR)

(21) Appl. No.: 16/573,737

(22) Filed: Sep. 17, 2019

(30) Foreign Application Priority Data
Aug. 20, 2019 (KR) 10-2019-0101777

(51) Int. Cl. A61B 5/04; A61B 5/00; A61B 5/0408; A61B 5/18

(52) U.S. Cl. CPC A61B 5/04012; A61B 5/6893; A61B 5/0432; A61B 5/18; A61B 5/7232; A61B 5/0408

(57) ABSTRACT

According to an embodiment of the present disclosure, a vehicular electrocardiogram measurement device includes an electrocardiographic sensor which is installed in a seat of a vehicle and obtains a patch signal, and a controller, which, on the basis of the patch signal obtained by the electrocardiographic sensor, generates a first request signal for requesting contact of a user's body with a patch of the electrocardiographic sensor. At least one among an autonomous vehicle, a user terminal, or a server according to embodiments of the present disclosure may be combined or associated with an artificial intelligence module, an unmanned aerial vehicle (UAV), a robot, an augmented reality (AR) device, and a device related to virtual reality (VR) or 5G service.

### CROSS-REFERENCE TO RELATED APPLICATION

This present application claims benefit of priority to Korean Patent Application No. 10-2019-0101777, entitled “VEHICULAR ELECTROCARDIOGRAM MEASUREMENT DEVICE AND METHOD,” filed on Aug. 20, 2019, in the Korean Intellectual Property Office, the entire disclosure of which is incorporated herein by reference.

### BACKGROUND

### 1. Technical Field

The present disclosure relates to an electrocardiogram measurement technology, and more particularly, to a vehicular electrocardiogram measurement device and method for measuring an electrocardiogram in a limited environment such as a vehicle.

### 2. Description of Related Art

An electrocardiograph (ECG) technology is widely used in various fields to measure biometric signals of human beings. An electrocardiographic sensor which uses an electrocardiograph may be used to monitor a health condition of a driver in a vehicle environment.

Korean Patent Registration No. 1679976 discloses one of conventional methods for measuring an electrocardiogram of a driver in a vehicle environment. According to this method, a plurality of sensor units for measuring an electrocardiogram of a driver or occupant are provided, and the electrocardiogram is measured after checking whether the driver or occupant is in contact with the sensor units using a reference voltage obtained from a non-contact portion.

However, according to the conventional in-vehicle electrocardiographic device disclosed in the above document, only an error that occurs since a human body does not contact can be compensated for, and an error caused by movement or the thickness of clothes of a driver cannot be resolved, and moreover, an additional non-contact sensor for compensating for a non-contact error is required.

Therefore, an electrocardiogram cannot be accurately measured when a driver or occupant wears thick clothes or moves continuously.

Accordingly, a technology is required to be developed to accurately measure an electrocardiogram in consideration of clothes worn by a driver or movement of the driver.

### SUMMARY OF THE INVENTION

An aspect of the present disclosure is to provide a vehicular electrocardiogram measurement device and method based on a scheme of compensating for various errors such as an electrocardiogram measurement error due to movement or wearing of clothes by measuring a peak-to-peak voltage and a period thereof rather than based on a scheme, which is a cause of the above problem, of only compensating for an error due to faulty contact by using a non-contact reference voltage.

Another aspect of the present disclosure is to provide a vehicular electrocardiogram measurement device and method for addressing various error situations that may occur in an environment in a vehicle by using a unique characteristic of a signal, i.e., a peak-to-peak voltage, which may be measured by an electrocardiographic sensor, without installing an additional signal-compensating device.

The present disclosure is not limited to what has been described above, and other aspects not mentioned herein will be apparent from the following description to one of ordinary skill in the art to which the present disclosure pertains.

To resolve the above problems, a vehicular electrocardiogram measurement device according to an embodiment of the present disclosure may measure an electrocardiogram on the basis of the magnitude and period of a peak-to-peak voltage of a signal obtained using an electrocardiographic sensor installed in a seat of a vehicle.

In detail, an embodiment of the present disclosure may be a vehicular electrocardiogram measurement device including an electrocardiographic sensor which is installed in a seat of a vehicle and obtains a patch signal, and a controller, which, on the basis of the patch signal obtained by the electrocardiographic sensor, generates a first request signal for requesting contact of a user's body with a patch of the electrocardiographic sensor if a first condition is not satisfied, generates a second request signal for requesting to take off clothes if a second condition is not satisfied, generates a third request signal for requesting to stop moving if a third condition is not satisfied, and determines a stress level on the basis of the patch signal as the first condition, the second condition, and the third condition are satisfied, wherein the first condition is that an absolute value of a voltage of the patch signal exceeds a predetermined first voltage value, the second condition is that a peak-to-peak voltage value of the patch signal exceeds a predetermined second voltage value, and the third condition is that the peak-to-peak voltage value of the patch signal is measured periodically.

In an embodiment of the present disclosure, the electrocardiographic sensor may be activated in response to starting of the vehicle, and the controller may obtain the patch signal when a predetermined time has elapsed since a seat belt of the seat of the vehicle was fastened.

In an embodiment of the present disclosure, the controller may periodically calculate a heart rate on the basis of the patch signal, and may determine the stress level on the basis of the patch signal when an absolute value of a difference between the heart rate of a current period and the heart rate of a previous period is at least a predetermined value.

In an embodiment of the present disclosure, the vehicular electrocardiogram measurement device may further include a communicator, wherein the communicator may receive compensation data for setting the first voltage value and second voltage value on the basis of a downlink grant of a 5G network connected to drive in an automatic driving mode.

In an embodiment of the present disclosure, the driving mode may include a defensive automatic driving mode and an aggressive automatic driving mode, the controller may generate a control signal for requesting a switch from the defensive automatic driving mode to the aggressive automatic driving mode when the stress level determined during driving in the defensive automatic driving mode is at least a predetermined reference level, and the communicator may transmit the control signal on the basis of an uplink grant of the 5G network connected to drive in the automatic driving mode.

In an embodiment of the present disclosure, the vehicular electrocardiogram measurement device may further include a user interface, wherein the automatic driving mode may include a defensive automatic driving mode and an aggressive automatic driving mode, the controller may generate a control signal for requesting a switch from the defensive automatic driving mode to the aggressive automatic driving mode when the stress level determined during driving in the defensive automatic driving mode is at least a predetermined reference level and a signal for requesting the switch from the defensive automatic driving mode to the aggressive automatic driving mode is received via the user interface, and the communicator may transmit the control signal on the basis of an uplink grant of the 5G network connected to drive in the automatic driving mode.

An embodiment of the present disclosure may be a vehicular electrocardiogram measurement device connected to a vehicle including an electrocardiographic sensor which is installed in a seat and is activated in response to starting of the vehicle and obtains a patch signal, the vehicular electrocardiographic measurement device including a controller, which, on the basis of the patch signal obtained by the electrocardiographic sensor, generates a first request signal for requesting contact of a user's body with a patch of the electrocardiographic sensor if a first condition is not satisfied, generates a second request signal for requesting to take off clothes if a second condition is not satisfied, generates a third request signal for requesting to stop moving if a third condition is not satisfied, and determines a stress level on the basis of the patch signal as the first condition, the second condition, and the third condition are satisfied, wherein the first condition is that an absolute value of a voltage of the patch signal is less than a predetermined first voltage value, the second condition is that a peak-to-peak voltage value of the patch signal is at least a predetermined second voltage value, and the third condition is that the peak-to-peak voltage value of the patch signal is measured periodically.

In an embodiment of the present disclosure, the controller may obtain the patch signal when elapse of a predetermined time is recognized after a seat belt of the seat of the vehicle is fastened.

In an embodiment of the present disclosure, the controller may periodically calculate a heart rate on the basis of the patch signal, and may determine the stress level on the basis of the patch signal when an absolute value of a difference between the heart rate of a current period and the heart rate of a previous period is at least a predetermined value.

An embodiment of the present disclosure may be a vehicular electrocardiogram measurement method including obtaining a patch signal through an electrocardiographic sensor installed in a seat of a vehicle, and on the basis of the patch signal obtained by the electrocardiographic sensor, generating, a first request signal for requesting contact of a user's body with a patch of the electrocardiographic sensor if a first condition is not satisfied, generating a second request signal for requesting to take off clothes if a second condition is not satisfied, generating a third request signal for requesting to stop moving if a third condition is not satisfied, and determining a stress level on the basis of the patch signal as the first condition, the second condition, and the third condition are satisfied, wherein the first condition is that an absolute value of a voltage of the patch signal exceeds a predetermined first voltage value, the second condition is that a peak-to-peak voltage value of the patch signal exceeds a predetermined second voltage value, and the third condition is that the peak-to-peak voltage value of the patch signal is measured periodically.

In an embodiment of the present disclosure, the vehicular electrocardiogram measurement method may further include activating the electrocardiographic sensor in response to starting of the vehicle, wherein the obtaining the patch signal may include obtaining the patch signal through the electrocardiographic sensor when a predetermined time has elapsed since a seat belt of the seat of the vehicle was fastened.

In an embodiment of the present disclosure, the vehicular electrocardiogram measurement method may further include periodically calculating a heart rate on the basis of the patch signal, and determining the stress level on the basis of the patch signal when an absolute value of a difference between the heart rate of a current period and the heart rate of a previous period is at least a predetermined value.

In an embodiment of the present disclosure, the vehicular electrocardiogram measurement method may further include receiving compensation data for setting the first voltage value and the second voltage value on the basis of a downlink grant of a 5G network connected to drive in an automatic driving mode.

In an embodiment of the present disclosure, the vehicular electrocardiogram measurement method may further include generating a control signal for requesting a switch from a defensive automatic driving mode to an aggressive automatic driving mode when the stress level determined during driving in the defensive automatic driving mode is at least a predetermined reference level, and transmitting the control signal on the basis of an uplink grant of the 5G network connected to drive in the automatic driving mode, wherein the automatic driving mode may include the defensive automatic driving mode and the aggressive automatic driving mode.

In an embodiment of the present disclosure, the vehicular electrocardiogram measurement method may further include generating a control signal for requesting a switch from a defensive automatic driving mode to an aggressive automatic driving mode when the stress level determined during driving in the defensive automatic driving mode is at least a predetermined reference level and a signal for requesting the switch from the defensive automatic driving mode to the aggressive automatic driving mode is received, and transmitting the control signal on the basis of an uplink grant of the 5G network connected to drive in the automatic driving mode, wherein the automatic driving mode comprises the defensive automatic driving mode and the aggressive automatic driving mode.

An embodiment of the present disclosure may be a computer-readable recording medium in which a vehicular electrocardiogram measurement program is recorded, the computer-readable recording medium including a means for obtaining a patch signal through an electrocardiographic sensor installed in a seat of a vehicle, and a means for, on the basis of the patch signal, generating a first request signal for requesting contact of a user's body with a patch of the electrocardiographic sensor if a first condition is not satisfied, generating a second request signal for requesting to take off clothes if a second condition is not satisfied, generating a third request signal for requesting to stop moving if a third condition is not satisfied, and determining a stress level on the basis of the patch signal as the first condition, the second condition, and the third condition are satisfied, wherein the first condition is that an absolute value of a voltage of the patch signal exceeds a predetermined first voltage value, the second condition is that a peak-to-peak voltage value of the patch signal exceeds a predetermined second voltage value, and the third condition is that the peak-to-peak voltage value of the patch signal is measured periodically.

Details about other embodiments are included in the detailed description and the drawings.

Embodiments of the present disclosure are not limited to the embodiments described above, and other embodiments not mentioned above will be clearly understood from the description below.

### BRIEF DESCRIPTION OF THE DRAWINGS

The above and other aspects, features, and advantages of the present disclosure will become apparent from the detailed description of the following aspects in conjunction with the accompanying drawings, in which:

FIG. 1 is a diagram illustrating a system in which a vehicular electrocardiogram measurement device according to an embodiment of the present disclosure is applied;

FIGS. 2 to 4 are diagrams illustrating vehicular electrocardiogram measurement devices according to embodiments of the present disclosure;

FIG. 5 is a graph illustrating an operation principle of a vehicular electrocardiogram measurement device according to an embodiment of the present disclosure;

FIG. 6 is a diagram illustrating an example of basic operations of an autonomous vehicle and a 5G network in a 5G communication system;

FIG. 7 is a diagram illustrating an example of operations of an autonomous vehicle and a 5G network in a 5G communication system;

FIGS. 8 to 11 are diagrams illustrating operation of an autonomous vehicle using 5G communication; and

FIG. 12 is an operation flowchart illustrating a vehicular electrocardiogram measurement method according to an embodiment of the present disclosure.

### DETAILED DESCRIPTION

Hereinafter, embodiments of the present disclosure will be described in detail with reference to the accompanying drawings.

Like reference numerals refer to the like elements throughout and a duplicate description thereof is omitted. Suffixes “module” and “unit or portion” for elements used in the following description are merely provided for facilitation of preparing this specification, and thus they are not granted a specific meaning or function. In the following description of the embodiments disclosed herein, the detailed description of related known technology will be omitted when it may obscure the subject matter of the embodiments according to the present disclosure. The accompanying drawings are merely used to help easily understand embodiments of the present disclosure, and it should be understood that the technical idea of the present disclosure is not limited by the accompanying drawings, and these embodiments include all changes, equivalents or alternatives within the idea and the technical scope of the present disclosure.

It will be understood that, although the terms “first”, “second”, and the like may be used herein to describe various elements, these elements should not be limited by these terms. These terms are generally only used to distinguish one element from another.

When an element or layer is referred to as being “on,” “engaged to,” “connected to,” or “coupled to” another element or layer, it may be directly on, engaged, connected, or coupled to the other element or layer, or intervening elements or layers may be present. The terms “connected” and “coupled” are not restricted to physical or mechanical connections or couplings, and can include electrical connections or couplings, whether direct or indirect.

The connection can be such that the objects are permanently connected or releasably connected.

It must be noted that as used herein and in the appended claims, the singular forms “a,” “an,” and “the” include the plural references unless the context clearly dictates otherwise.

It should be understood that the terms “comprises,” “comprising,” “includes,” “including,” “containing,” “has,” “having” or any other variation thereof specify the presence of stated features, integers, steps, operations, elements, and/or components, but do not preclude the presence or addition of one or more other features, integers, steps, operations, elements, and/or components.

A vehicle described in this specification refers to a car, a motorcycle, and the like. Hereinafter, the vehicle will be exemplified as a car.

The vehicle described in the specification may include, but is not limited to, a vehicle having an internal combustion engine as a power source, a hybrid vehicle having an engine and an electric motor as a power source, and an electric vehicle having an electric motor as a power source.

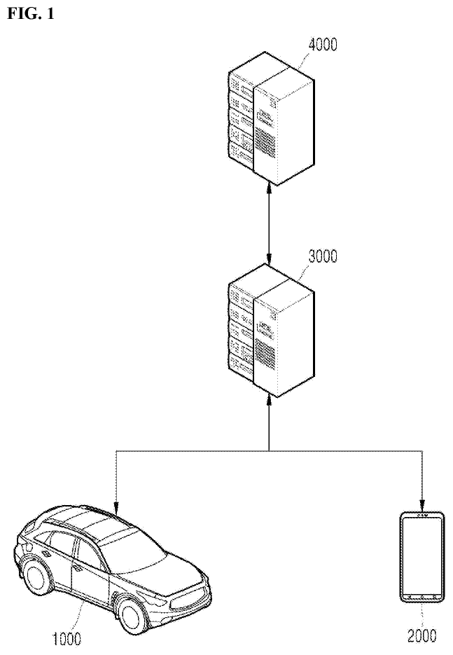

FIG. 1 is a diagram illustrating a system in which a vehicular electrocardiogram measurement device according to an embodiment of the present disclosure is applied.

Referring to FIG. 1, vehicular electrocardiogram measurement devices 1000,2000 may be a device 1000 installed in a vehicle or a device 2000 installed in a user terminal.

The vehicular electrocardiogram measurement device 1000 installed in a vehicle may determine a stress level using a signal acquired from an electrocardiographic sensor mounted on a vehicle seat, and may transmit the determined stress level to the user terminal 2000 or a server 3000.

The vehicular electrocardiogram measurement device 2000 installed in a user terminal may acquire an electrocardiographic signal by communicating with the electrocardiographic sensor mounted on the vehicle seat, may determine a stress level using the acquired signal, and may transmit the determined stress level to the server 3000.

The server 3000 may transfer, to an external server 4000 such as a medical institution server, the stress levels received from the vehicular electrocardiogram measurement devices 1000, 2000.

The stress level information transferred to the medical institution server may be used as analysis data when an occupant visits a medical institution.

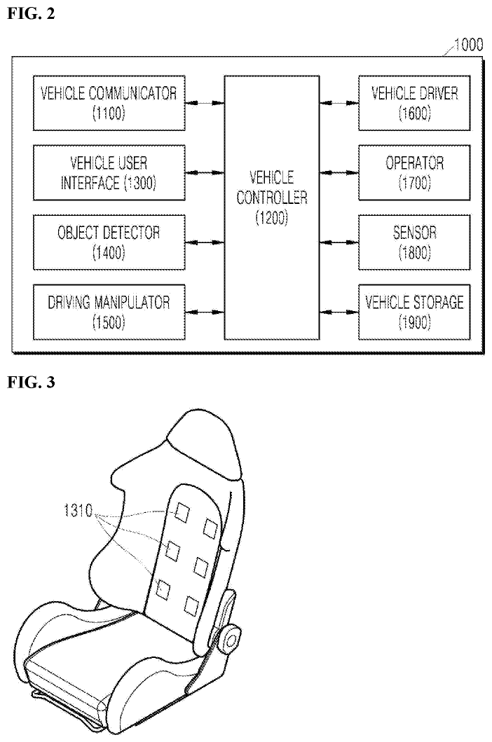

FIGS. 2 to 4 are diagrams illustrating vehicular electrocardiogram measurement devices according to embodiments of the present disclosure.

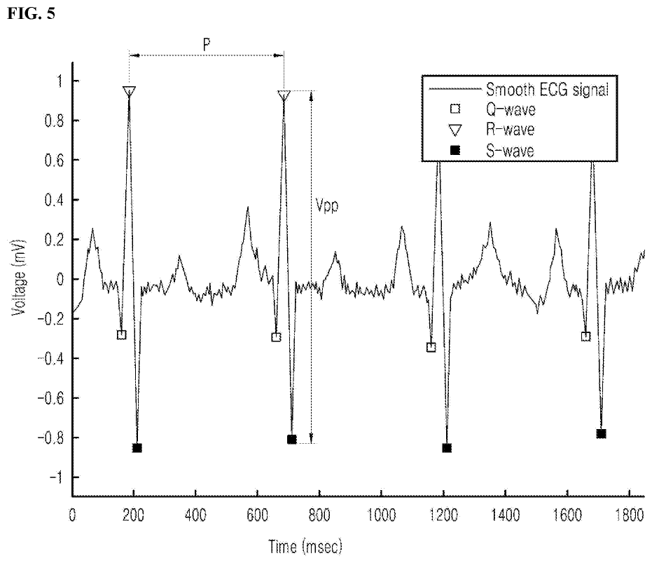

FIG. 5 is a graph illustrating an operation principle of a vehicular electrocardiogram measurement device according to an embodiment of the present disclosure.

Referring to FIG. 2, the vehicular electrocardiogram measurement device 1000 installed in a vehicle may include a vehicle communicator 1100, a vehicle controller 1200, a vehicle user interface 1300, an object detector 1400, a driving manipulator 1500, a vehicle driver 1600, an operator 1700, a sensor 1800, and a vehicle storage 1900.

According to an embodiment, the vehicular electrocardiogram measurement device may include other elements in addition to the elements illustrated in FIG. 2 and described below, or may not include portion of the elements illustrated in FIG. 2 and described below.

The vehicle communicator 1100 may be a module for performing communication with an external device. Herein, the external device may be the user terminal or the server 3000.

The vehicle communicator 1100 may receive compensation data for setting a threshold value, i.e., a first voltage value and a second voltage value, which is a criterion for normal measurement of an electrocardiographic sensor 1310, on the basis of a downlink grant of a 5G network connected to operate in an automatic driving mode.

The vehicle communicator 1100 may transmit a control signal for requesting a switch from a defensive automatic driving mode to an aggressive automatic driving mode on the basis of an uplink grant of a 5G network connected to operate in an automatic driving mode.

A vehicle in which the vehicular electrocardiogram measurement device 1000 is installed may be switched from the automatic driving mode to a manual mode or vice versa according to a driving situation. Here, the driving situation may be determined on the basis of information received by the vehicle communicator 1100.

The vehicle communicator 1100 may include at least one among a transmission antenna, a reception antenna, a radio frequency (RF) circuit capable of implementing various communication protocols, and an RF element in order to perform communication.

The vehicle communicator 1100 may perform short-range communication, GPS signal reception, V2X communication, optical communication, broadcast transmission/reception, and intelligent transport systems (ITS) communication function.

The vehicle communicator 1100 may further support other functions than the functions described, or may not support some of the functions described, depending on the embodiment.

The vehicle communicator 1100 may support short-range communication by using at least one among Bluetooth™, Radio Frequency Identification (RFID), Infrared Data Association (IrDA), Ultra WideBand (UWB), ZigBee, Near Field Communication (NFC), Wireless-Fidelity (Wi-Fi), Wi-Fi Direct, and Wireless Universal Serial Bus (Wireless USB) technologies.

The vehicle communicator 1100 may form short-range wireless communication networks so as to perform short-range communication between the vehicle and at least one external device.

The vehicle communicator 1100 may include a Global Positioning System (GPS) module or a Differential Global Positioning System (DGPS) module for obtaining location information of the vehicle.

The vehicle communicator 1100 may include a module for supporting wireless communication between the vehicle and a server (V2I: vehicle to infrastructure), communication with another vehicle (V2V: vehicle to vehicle) or communication with a pedestrian (V2P: vehicle to pedestrian). That is, the vehicle communicator 1100 may include a V2X communication module. The V2X communication module may include an RF circuit capable of implementing V2I, V2V, and V2P communication protocols.

The vehicle communicator 1100 may receive a danger information broadcast signal transmitted by another vehicle through the V2X communication module, and may transmit a danger information inquiry signal and receive a danger information response signal in response thereto.

The vehicle communicator 1100 may include an optical communication module for performing communication with an external device via light. The optical communication module may include both a light transmitting module for converting electrical signals into optical signals and transmitting the optical signals to the outside, and a light receiving module for converting the received optical signals into electrical signals.

According to an embodiment, the light transmitting module may be integrally formed with a lamp included in the vehicle.

The vehicle communicator 1100 may include a broadcast communication module for receiving broadcast signals from an external broadcast management server, or transmitting broadcast signals to the broadcast management server through broadcast channels. Examples of the broadcast channels may include a satellite channel and a terrestrial channel. Example of the broadcast signal may include a TV broadcast signal, a radio broadcast signal, and a data broadcast signal.

The vehicle communicator 1100 may include an ITS communication module that exchanges information, data or signals with a traffic system. The ITS communication module may provide the obtained information and data to the traffic system. The ITS communication module may receive information, data, or signals from the traffic system. For example, the ITS communication module may receive road traffic information from the communication system and provide the road traffic information to the vehicle controller 1200. For example, the ITS communication module may receive control signals from the traffic system and provide the control signals to the vehicle controller 1200 or a processor provided in the vehicle.

Depending on the embodiment, the overall operation of each module of the vehicle communicator 1100 may be controlled by a separate process provided in the vehicle communicator 1100. The vehicle communicator 1100 may include a plurality of processors, or may not include a processor. When a processor is not included in the vehicle communicator 1100, the vehicle communicator 1100 may be operated by either a processor of another apparatus in the vehicle or the vehicle controller 1200.

The vehicle communicator 1100 may, together with the vehicle user interface 1300, implement a vehicle-use display device. In this case, the vehicle-use display device may be referred to as a telematics device or an audio video navigation (AVN) device.

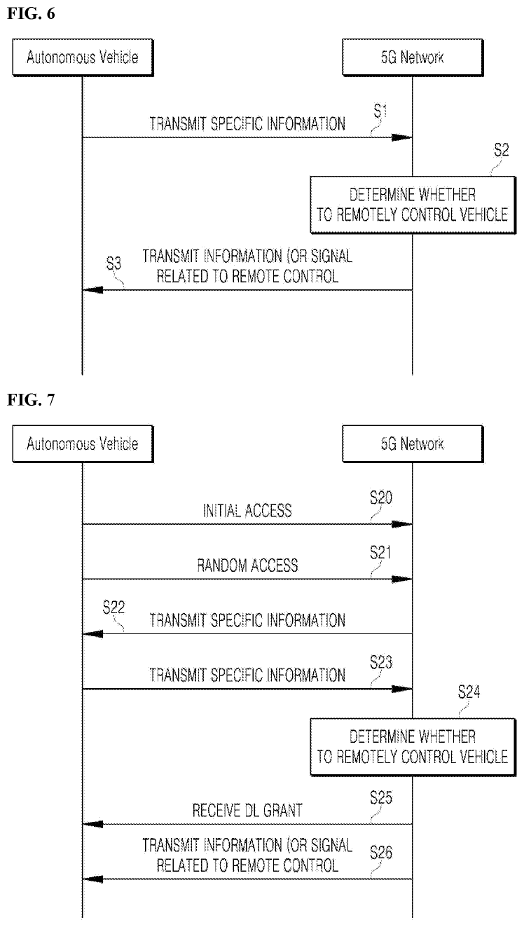

FIG. 6 is a diagram showing an example of the basic operation of an autonomous vehicle and a 5G network in a 5G communication system.

The vehicle communicator 1100 may transmit specific information over a 5G network when the vehicle is operated in the autonomous driving mode.

The specific information may include autonomous driving related information.

The autonomous driving related information may be information directly related to the driving control of the vehicle. For example, the autonomous driving related information may include at least one among object data indicating an object near the vehicle, map data, vehicle status data, vehicle location data, and driving plan data.

The autonomous driving related information may further include service information necessary for autonomous driving. For example, the specific information may include information on a destination inputted through the user terminal 1300 and a safety rating of the vehicle.

In addition, the 5G network may determine whether a vehicle is to be remotely controlled (S2).

The 5G network may include a server or a module for performing remote control related to autonomous driving.

The 5G network may transmit information (or a signal) related to the remote control to an autonomous driving vehicle (S3).

As described above, information related to the remote control may be a signal directly applied to the autonomous driving vehicle, and may further include service information necessary for autonomous driving. The autonomous driving vehicle according to this embodiment may receive service information such as insurance for each interval selected on a driving route and risk interval information, through a server connected to the 5G network to provide services related to the autonomous driving.

An essential process for performing 5G communication between the autonomous driving vehicle and the 5G network (for example, an initial access process between the vehicle and the 5G network) will be briefly described with reference to FIG. 7 to FIG. 11 below.

An example of operations through the autonomous driving vehicle performed in the 5G communication system and the 5G network is as follows.

The vehicle may perform an initial access process with the 5G network (initial access step, S20). The initial access process may include a cell search process for downlink (DL) synchronization acquisition and a process for obtaining system information.

The vehicle may perform a random access process with the 5G network (random access step, S21). The random access process may include a process for uplink (UL) synchronization acquisition or a preamble transmission process for UL data transmission, or a random access response receiving process.

The 5G network may transmit an Uplink (UL) grant for scheduling transmission of specific information to the autonomous driving vehicle (UL grant receiving step, S22).

The process in which the vehicle receives the UL grant may include a scheduling process for being assigned a time/frequency resource for the transmission of the UL data over the 5G network.

The autonomous driving vehicle may transmit specific information over the 5G network based on the UL grant (specific information transmission step, S23).

The 5G network may determine whether the vehicle is to be remotely controlled based on the specific information transmitted from the vehicle (vehicle remote control determination step, S24).

The autonomous driving vehicle may receive the DL grant through a physical DL control channel for receiving a response on pre-transmitted specific information from the 5G network (DL grant receiving step, S25).

The 5G network may transmit information (or a signal) related to the remote control to the autonomous driving vehicle based on the DL grant (remote control related information transmission step, S26).

A process in which the initial access process and/or the random access process between the 5G network and the autonomous driving vehicle is combined with the DL grant receiving process has been exemplified. However, the present disclosure is not limited thereto.

For example, the initial access process and/or the random access process may be performed through the initial access step, the UL grant receiving step, the specific information transmission step, the vehicle remote control determination step, and the remote control related information transmission step. In addition, for example, the initial access process and/or the random access process may be performed through the random access step, the UL grant receiving step, the specific information transmission step, the vehicle remote control determination step, and the remote control related information transmission step. The autonomous driving vehicle may be controlled by the combination of an AI operation and the DL grant receiving process through the specific information transmission step, the vehicle remote control determination step, the DL grant receiving step, and the remote control related information transmission step.

The operation of the autonomous driving vehicle described above is merely exemplary, but the present disclosure is not limited thereto.

For example, the operation of the autonomous driving vehicle may be performed by selectively combining the initial access step, the random access step, the UL grant receiving step, or the DL grant receiving step with the specific information transmission step, or the remote control related information transmission step. The operation of the autonomous driving vehicle may include the random access step, the UL grant receiving step, the specific information transmission step, and the remote control related information transmission step. The operation of the autonomous driving vehicle may include the initial access step, the random access step, the specific information transmission step, and the remote control related information transmission step. The operation of the autonomous driving vehicle may include the UL grant receiving step, the specific information transmission step, the DL grant receiving step, and the remote control related information transmission step.

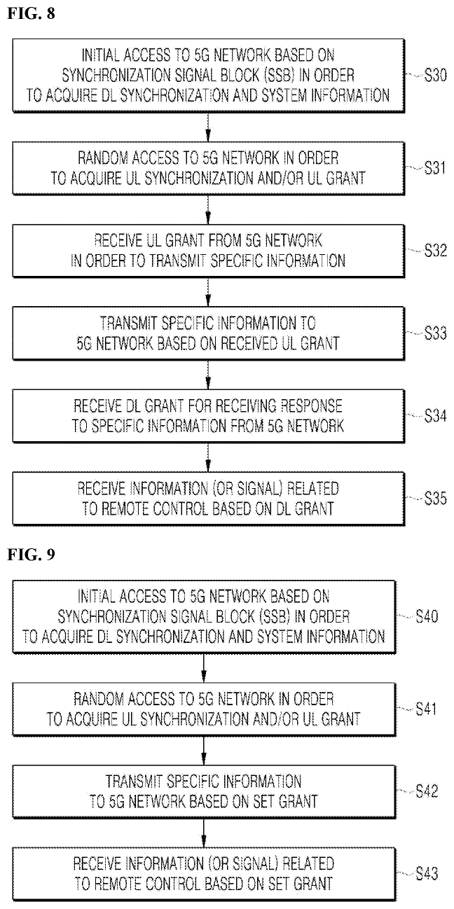

As illustrated in FIG. 8, the vehicle including an autonomous driving module may perform an initial access process with the 5G network based on Synchronization Signal Block (SSB) in order to acquire DL synchronization and system information (initial access step).

The autonomous driving vehicle may perform a random access process with the 5G network for UL synchronization acquisition and/or UL transmission (random access step, S31).

The autonomous driving vehicle may receive the UL grant from the 5G network for transmitting specific information (UL grant receiving step, S32).

The autonomous driving vehicle may transmit the specific information to the 5G network based on the UL grant (specific information transmission step, S33).

The autonomous driving vehicle may receive the DL grant from the 5G network for receiving a response to the specific information (DL grant receiving step, S34).

The autonomous driving vehicle may receive remote control related information (or a signal) from the 5G network based on the DL grant (remote control related information receiving step, S35).

A beam management (BM) process may be added to the initial access step, and a beam failure recovery process associated with Physical Random Access Channel (PRACH) transmission may be added to the random access step. QCL (Quasi Co-Located) relation may be added with respect to the beam reception direction of a Physical Downlink Control Channel (PDCCH) including the UL grant in the UL grant receiving step, and QCL relation may be added with respect to the beam transmission direction of the Physical Uplink Control Channel (PUCCH)/Physical Uplink Shared Channel (PUSCH) including specific information in the specific information transmission step. In addition, QCL relation may be added with respect to the beam reception direction of the PDCCH including the DL grant in the DL grant receiving step.

As illustrated in FIG. 9, the autonomous driving vehicle may perform an initial access process with the 5G network based on SSB for acquiring DL synchronization and system information (initial access step, S40).

The autonomous driving vehicle may perform a random access process with the 5G network for UL synchronization acquisition and/or UL transmission (random access step, S41).

The autonomous driving vehicle may transmit specific information based on a configured grant to the 5G network (UL grant receiving step, S42). In other words, the autonomous driving vehicle 1000 may receive the configured grant instead of receiving the UL grant from the 5G network.

The autonomous driving vehicle may receive the remote control related information (or a signal) from the 5G network based on the configured grant (remote control related information receiving step, S43).

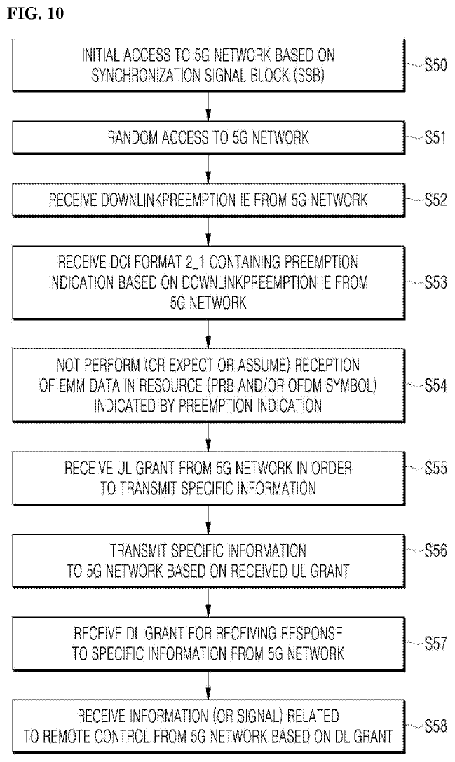

As illustrated in FIG. 10, the autonomous driving vehicle may perform an initial access process with the 5G network based on SSB for acquiring DL synchronization and system information (initial access step, S50).

The autonomous driving vehicle may perform a random access process with the 5G network for UL synchronization acquisition and/or UL transmission (random access step, S51).

In addition, the autonomous driving vehicle may receive Downlink Preemption (DL) and Information Element (IE) from the 5G network (DL Preemption IE reception step, S52).

The autonomous driving vehicle may receive DCI (Downlink Control Information) format 2_1 including preemption indication based on the DL preemption IE from the 5G network (DCI format 2_1 receiving step, S53).

The autonomous driving vehicle may not perform (or expect or assume) the reception of eMBB data in the resource (PRB and/or OFDM symbol) indicated by the pre-emption indication (step of not receiving eMBB data, S54).

The autonomous driving vehicle may receive the UL grant over the 5G network for transmitting specific information (UL grant receiving step, S55).

The autonomous driving vehicle may transmit the specific information to the 5G network based on the UL grant (specific information transmission step, S56).

The autonomous driving vehicle may receive the DL grant from the 5G network for receiving a response to the specific information (DL grant receiving step, S57).

The autonomous driving vehicle may receive the remote control related information (or signal) from the 5G network based on the DL grant (remote control related information receiving step, S58).

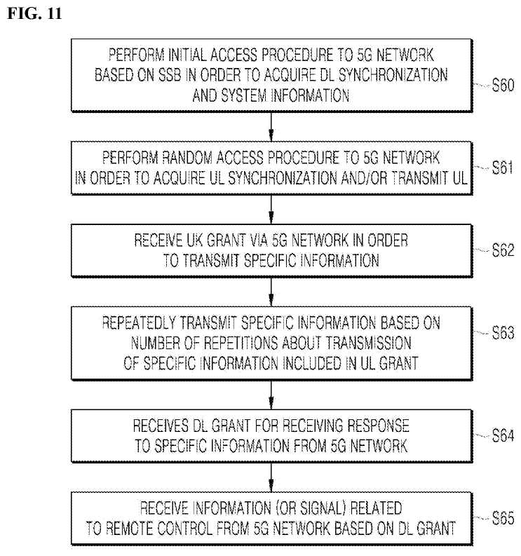

As illustrated in FIG. 11, the autonomous driving vehicle may perform an initial access process with the 5G network based on SSB for acquiring DL synchronization and system information (initial access step, S60).

The autonomous driving vehicle may perform a random access process with the 5G network for UL synchronization acquisition and/or UL transmission (random access step, S61).

The autonomous driving vehicle may receive the UL grant over the 5G network for transmitting specific information (UL grant receiving step, S62).

When specific information is transmitted repeatedly, the UL grant may include information on the number of repetitions, and the specific information may be repeatedly transmitted based on information on the number of repetitions (specific information repetition transmission step, S63).

The autonomous driving vehicle may transmit the specific information to the 5G network based on the UL grant.

The repeated transmission of the specific information may be performed by frequency hopping, and the first transmission of the specific information may be performed from a first frequency resource, and the second transmission of the specific information may be performed from a second frequency resource.

The specific information may be transmitted through Narrowband of Resource Block (6RB) and Resource Block (1RB).

The autonomous driving vehicle may receive the DL grant from the 5G network for receiving a response to the specific information (DL grant receiving step, S64).

The autonomous driving vehicle may receive the remote control related information (or signal) from the 5G network based on the DL grant (remote control related information receiving step, S65).

The above-described 5G communication technique can be applied in combination with the embodiment proposed in this specification, which will be described in FIG. 1 to FIG. 12, or supplemented to specify or clarify the technical feature of the embodiment proposed in this specification.

The vehicle may be connected to an external server through a communication network, and may be capable of moving along a predetermined route without a driver's intervention by using an autonomous driving technique.

In the embodiment described below, a user may be interpreted as a driver, a passenger, or an owner of a user terminal.

While the vehicle is driving in the autonomous driving mode, the type and frequency of accident occurrence may depend on the capability of the vehicle of sensing dangerous elements in the vicinity in real time. The route to the destination may include intervals with different levels of risk based on various causes, such as weather, terrain characteristic, and traffic congestion.

At least one among an autonomous driving vehicle, a user terminal, and a server according to embodiments of the present disclosure may be associated or integrated with an artificial intelligence module, a drone (unmanned aerial vehicle (UAV)), a robot, an augmented reality (AR) device, a virtual reality (VR) device, a 5G service related device, and the like.

For example, the vehicle may operate in association with at least one artificial intelligence module or robot included in the vehicle in the autonomous driving mode.

For example, the vehicle may interact with at least one robot. The robot may be an autonomous mobile robot (AMR) capable of driving by itself. Being capable of driving by itself, the AMR may freely move, and may include a plurality of sensors so as to avoid obstacles during traveling. The AMR may be a flying robot (such as a drone) equipped with a flight device. The AMR may be a wheel-type robot equipped with at least one wheel, and which is moved through the rotation of the at least one wheel. The AMR may be a leg-type robot equipped with at least one leg, and which is moved using the at least one leg.

The robot may function as a device that enhances the convenience of a user of a vehicle. For example, the robot may move a load placed in the vehicle to a final destination. For example, the robot may perform a function of providing route guidance to a final destination to a user who alights from the vehicle. For example, the robot may perform a function of transporting the user who alights from the vehicle to the final destination

At least one electronic apparatus included in the vehicle may communicate with the robot through a communication device.

At least one electronic apparatus included in the vehicle may provide, to the robot, data processed by the at least one electronic apparatus included in the vehicle. For example, at least one electronic apparatus included in the vehicle may provide, to the robot, at least one among object data indicating an object near the vehicle, HD map data, vehicle status data, vehicle position data, and driving plan data.

At least one electronic apparatus included in the vehicle may receive, from the robot, data processed by the robot. At least one electronic apparatus included in the vehicle may receive at least one of sensing data, object data, robot status data, robot location data, or robot movement plan data which is generated by the robot.

At least one electronic apparatus included in the vehicle may generate a control signal further based on data received from the robot. For example, at least one electronic apparatus included in the vehicle may compare information on the object generated by an object detection device with information on the object generated by the robot, and generate a control signal based on the comparison result. At least one electronic apparatus included in the vehicle may generate a control signal so that interference between the vehicle movement route and the robot movement route may not occur.

At least one electronic apparatus included in the vehicle may include a software module or a hardware module for implementing an artificial intelligence (AI) (hereinafter referred to as an artificial intelligence module). At least one electronic apparatus included in the vehicle 1000 may input obtained data into the artificial intelligence module, and use data outputted from the artificial intelligence module.

The artificial intelligence module may perform machine learning of input data by using at least one artificial neural network (ANN). The artificial intelligence module may output driving plan data through machine learning of input data.

At least one electronic apparatus included in the vehicle may generate a control signal based on the data processed by the artificial intelligence.

According to the embodiment, at least one electronic apparatus included in the vehicle may receive data processed by an artificial intelligence from an external device through a communication device. At least one electronic apparatus included in the vehicle may generate a control signal based on the data processed by the artificial intelligence.

On the basis of a patch signal obtained by the electrocardiographic sensor 1310, the vehicle controller 1200 may generate a first request signal for requesting contact of a user's body with a patch of the electrocardiographic sensor 1310 if a first condition is not satisfied, may generate a second request signal for requesting to take off clothes if a second condition is not satisfied, may generate a third request signal for requesting to stop moving if a third condition is not satisfied, and may determine a stress level on the basis of a patch signal as the first to third conditions are satisfied.

Here, the first condition may be that an absolute value of a voltage of the patch signal exceeds a predetermined first voltage value, the second condition may be that a peak-to-peak voltage value of the patch signal exceeds a predetermined second voltage value, and the third condition may be that the peak-to-peak voltage value of the patch signal is measured periodically.

The vehicle controller 1200 may determine whether a body of an occupant is in contact with the electrocardiographic sensor 1310 on the basis of a threshold value for differentiating on/off of current.

The vehicle controller 1200 may determine that contact is faulty (current off) when an absolute value of a voltage of a wave obtained through the electrocardiographic sensor 1310 remains at a value equal to or smaller than a first voltage value that is a predetermined threshold value for determining on/of, for example, about 0.1 mV, during a certain period of time, for example, about five seconds.

The vehicle controller 1200 may determine that contact is established (current on) when there is a period in which the absolute value of the voltage of the wave obtained through the electrocardiographic sensor 1310 exceeds the first voltage value that is a predetermined threshold value for determining on/of, for example, about 0.1 mV, during the certain period of time, for example, about five seconds.

The vehicle controller 1200 may receive compensation data for determining or updating the first voltage value from the server 3000 via the vehicle communicator 1100, and may set the first voltage value using the received compensation data.

When it is determined that contact is faulty, the vehicle controller 1200 may generate the first request signal for inducing contact of a body with the patch of the electrocardiographic sensor 1310, and may provide the generated first request signal to the user interface 1300.

The vehicle controller 1200 may determine, on the basis of a magnitude of a peak-to-peak voltage Vpp of the wave obtained through the electrocardiographic sensor 1310, whether normal signal acquisition of the electrocardiographic sensor 1310 is possible when the occupant wears clothes, i.e., whether it is necessary to take off the clothes to obtain an electrocardiographic signal due to the thickness of the clothes of the occupant.

Referring to FIG. 5, the peak-to-peak voltage value is a voltage value corresponding to a difference between an R peak value (R-wave) and an S peak value (S-wave) observed from a typical wave of an electrocardiographic signal, wherein the S peak value may be measured within about 0.1 second after occurrence of the R peak value.

The vehicle controller 1200 may determine that it is necessary to take off clothes when the peak-to-peak voltage value of the wave obtained through the electrocardiographic sensor 1310 is equal to or smaller than a second voltage value Vtpp (Vthreshold peak-to-peak), for example, about 1.5 mV, that is a predetermined threshold value for determining whether to request to take off clothes.

The vehicle controller 1200 may determine that it is not necessary to take off clothes when the peak-to-peak voltage value of the wave obtained through the electrocardiographic sensor 1310 exceeds a second voltage value, for example, about 1.5 mV, that is a predetermined threshold value for determining whether to request to take off clothes.

The vehicle controller 1200 may receive compensation data for determining or updating the second voltage value from the server 3000 via the vehicle communicator 1100, and may set the second voltage value using the received compensation data.

When it is determined that taking off clothes is necessary, the vehicle controller 1200 may generate the second request signal for inducing the occupant to take off clothes, and may provide the generated second request signal to the user interface 1300.

The vehicle controller 1200 may determine, on the basis of a period of the peak-to-peak voltage value of the wave obtained through the electrocardiographic sensor 1310, whether normal signal acquisition of the electrocardiographic sensor 1310 for measuring a stress level is possible, i.e., whether it is necessary to stop movement of the occupant.

Referring to FIG. 5, the peak-to-peak voltage value Vpp which exceeds the second voltage value Vtpp is required to be generated at a predetermined period P in order to measure an electrocardiogram normally using the patch signal obtained by the electrocardiographic sensor 1310.

The vehicle controller 1200 may determine that movement of the occupant is required to be stopped when the period at which the peak-to-peak voltage value of the wave obtained through the electrocardiographic sensor 1310 is generated is not constant during a certain period of time, such as about 30 seconds, for example when a 1-sigma interval of a normal distribution curve obtained by measuring and normalizing a generation time interval of the peak-to-peak voltage value exceeds about 30%.

The vehicle controller 1200 may refer to a typical period at which the peak-to-peak voltage value of the wave obtained through the electrocardiographic sensor 1310 is generated, so as to determine that movement of the occupant is required to be stopped when the generation time interval of the peak-to-peak voltage value exceeds about two seconds or is equal to or shorter than about 0.3 seconds during a certain period of time, such as about 30 seconds.

The vehicle controller 1200 may determine that movement of the occupant is required to be stopped when the number of occurrences of the peak-to-peak voltage value of the wave obtained through the electrocardiographic sensor 1310 is equal to or less than about 30 times or exceeds about 200 times during a certain period of time, such as about 30 seconds.

The vehicle controller 1200 may determine that movement of the occupant is not required to be stopped when the period at which the peak-to-peak voltage of the wave obtained through the electrocardiographic sensor 1310 is generated is constant during a certain period of time, such as about 30 seconds, for example when the 1-sigma interval of the normal distribution curve obtained by measuring and normalizing the generation time interval of the peak-to-peak voltage value is about 30% or less.

The vehicle controller 1200 may refer to a typical period at which the peak-to-peak voltage value of the wave obtained through the electrocardiographic sensor 1310 is generated, so as to determine that movement of the occupant is not required to be stopped when the generation time interval of the peak-to-peak voltage value exceeds about 0.3 seconds and is equal to or shorter than about two seconds during a certain period of time, such as about 30 seconds.

The vehicle controller 1200 may determine that movement of the occupant is not required to be stopped when the number of occurrences of the peak-to-peak voltage value of the wave obtained through the electrocardiographic sensor 1310 exceeds about 30 times and is equal to or smaller than about 200 times during a certain period of time, such as about 30 seconds.

As the first condition, the second condition, and the third condition are satisfied, the vehicle controller 1200 may detect a peak value of a QRS wave composite signal through the electrocardiographic sensor 1310, may calculate a heart rate of the occupant on the basis of a time interval between peak values, and may detect an autonomic nervous system balance and a stress index (SI), described as below, of a person being measured, i.e., may determine a stress level by analyzing the heart rate calculated from the peak values of an electrocardiogram by applying a heart rate variability (HRV) analysis technology.

TABLE 1

| | Stress index | |
|---|---|---|
| Level | (SI) | Meaning |
| 1 | 25 or less | Almost no stress |
| 2 | 25-35 | Temporarily under stress |
| 3 | 35-45 | Initial stress |
| 4 | 45-60 | Temporary stress repeats |
| | | and stress tolerance begins |
| | | to weaken |
| 5 | 60 or more | Progressing to chronic stress |

The vehicle controller 1200 may detect whether a seat belt of a vehicle is fastened, and may obtain the patch signal from the electrocardiographic sensor 1310 when a predetermined time, for example, about five seconds, has elapsed since the seat belt was fastened, according to a result of the detection. Here, the time between when the seat belt is fastened and when the patch signal is obtained may be determined in consideration of a time required for an automatic vehicle function check and stabilization of the occupant, who is the driver, after the seat belt is fastened.

After initially determining a stress level, the vehicle controller 1200 may periodically calculate a heart rate on the basis of the patch signal obtained from the electrocardiographic sensor 1310, and may determine the stress level again on the basis of the patch signal when an absolute value of a difference between the heart rate of a current period and the heart rate of a previous period is at least a predetermined value, for example, about 10 times. That is, the vehicle controller 1200 may determine the stress level again to recognize an abnormal state of the occupant when an abnormal change in the heart rate is detected while driving the vehicle.

While driving the vehicle in the automatic driving mode including the defensive automatic driving mode and the aggressive automatic driving mode, the vehicle controller 1200 may generate a control signal for requesting a switch from the defensive automatic driving mode to the aggressive automatic driving mode and may transmit the generated control signal through the vehicle communicator 1100 when the current automatic driving mode is the defensive automatic driving mode and a determined stress level is at least a predetermined level, for example, level 3 (level 3 to level 5).

While driving the vehicle in the automatic driving mode including the defensive automatic driving mode and the aggressive automatic driving mode, the vehicle controller 1200 may confirm whether the driver desires to switch from the defensive automatic driving mode to the aggressive automatic driving mode through the user interface 1300 when the current automatic driving mode is the defensive automatic driving mode and a determined stress level is at least a predetermined level, for example, level 3 (level 3 to level 5). If the driver desires to switch from the defensive automatic driving mode to the aggressive automatic driving mode, the vehicle controller 1200 may generate a control signal for requesting a switch from the defensive automatic driving mode to the aggressive automatic driving mode, and may transmit the generated control signal through the vehicle communicator 1100.

The vehicle controller 1200 may recognize that the stress level increases when a driver having an aggressive driving tendency rides in a vehicle driven in the defensive automatic driving mode, and may request a switch from the defensive automatic driving mode to the aggressive automatic driving mode on the basis of an uplink grant of a 5G network connected to drive in the automatic driving mode via the vehicle communicator 1100.

The vehicle controller 1200 may generate a model for a driving tendency of each driver who rides in a vehicle using a machine learning algorithm on the basis of data collected by the sensor 1800, and may obtain the driving tendency of a driver who is currently riding based on the generate model and the data collected by the sensor 1800. Here, when the driver who is currently riding has an aggressive driving tendency, the vehicle controller 1200 may request to drive in the aggressive automatic driving mode via the vehicle communicator 1100.

Artificial intelligence (AI) is a field of computer engineering and information technology that researches a method for the computer to enable thinking, learning, self-development, etc. which are possible by human's intelligence, and means that the computer can imitate human's intelligent behavior.

In addition, the Artificial Intelligence does not exist in itself, but has many direct and indirect links with other fields of computer science. In recent years, there have been numerous attempts to introduce an element of AI into various fields of information technology to solve problems in the respective fields.

Machine Learning is a field of Artificial Intelligence, and a field of research that gives the ability capable of learning without an explicit program in the computer.

Specifically, the Machine Learning can be a technology for researching and constructing a system for learning, predicting, and improving its own performance based on empirical data and an algorithm for the same. The algorithms of the Machine Learning take a method of constructing a specific model in order to obtain the prediction or the determination based on the input data, rather than performing the strictly defined static program instructions.

Many Machine Learning algorithms have been developed on how to classify data in the Machine Learning. Decision Tree, Bayesian network, Support Vector Machine (SVM), Artificial Neural Network (ANN), etc. are representative examples.

The Decision Tree is an analytical method that performs classification and prediction by plotting a Decision Rule in a tree structure.

The Bayesian network is a model of the probabilistic relationship (conditional independence) between multiple variables in a graphical structure. The Bayesian network is suitable for data mining through Unsupervised Learning.

The Support Vector Machine is a model of Supervised Learning for pattern recognition and data analysis, and mainly used for classification and regression.

ANN is a data processing system modelled after the mechanism of biological neurons and interneuron connections, in which a number of neurons, referred to as nodes or processing elements, are interconnected in layers.

ANNs are models used in machine learning and may include statistical learning algorithms conceived from biological neural networks (particularly of the brain in the central nervous system of an animal) in machine learning and cognitive science.

ANNs may refer generally to models that have artificial neurons (nodes) forming a network through synaptic interconnections, and acquires problem-solving capability as the strengths of synaptic interconnections are adjusted throughout training.

The terms ‘artificial neural network’ and ‘neural network’ may be used interchangeably herein.

An ANN may include a number of layers, each including a number of neurons. In addition, the Artificial Neural Network can include the synapse for connecting between neuron and neuron.

An ANN may be defined by the following three factors: (1) a connection pattern between neurons on different layers; (2) a learning process that updates synaptic weights; and (3) an activation function generating an output value from a weighted sum of inputs received from a lower layer.

The Artificial Neural Network can include network models of the method such as Deep Neural Network (DNN), Recurrent Neural Network (RNN), Bidirectional Recurrent Deep Neural Network (BRDNN), Multilayer Perceptron (MLP), and Convolutional Neural Network (CNN), but is not limited thereto.

The terms “layer” and “hierarchy” may be used interchangeably herein.

An ANN may be classified as a single-layer neural network or a multi-layer neural network, based on the number of layers therein.

In general, a single-layer neural network may include an input layer and an output layer.

In addition, a general Multi-Layer Neural Network is composed of an Input layer, one or more Hidden layers, and an Output layer.

The Input layer is a layer that accepts external data, the number of neurons in the Input layer is equal to the number of input variables, and the Hidden layer is disposed between the Input layer and the Output layer and receives a signal from the Input layer to extract the characteristics to transfer it to the Output layer. The output layer receives a signal from the hidden layer and outputs an output value based on the received signal. The Input signal between neurons is multiplied by each connection strength (weight) and then summed, and if the sum is larger than the threshold of the neuron, the neuron is activated to output the output value obtained through the activation function.

Meanwhile, the Deep Neural Network including a plurality of Hidden layers between the Input layer and the Output layer can be a representative Artificial Neural Network that implements Deep Learning, which is a type of Machine Learning technology.

The Artificial Neural Network can be trained by using training data. Here, the training may refer to the process of determining parameters of the artificial neural network by using the training data, to perform tasks such as classification, regression analysis, and clustering of inputted data. Such parameters of the artificial neural network may include synaptic weights and biases applied to neurons.

An artificial neural network trained using training data can classify or cluster inputted data according to a pattern within the inputted data.

Throughout the present specification, an artificial neural network trained using training data may be referred to as a trained model.

Hereinbelow, learning paradigms of an artificial neural network will be described in detail.

Learning paradigms, in which an artificial neural network operates, may be classified into supervised learning, unsupervised learning, semi-supervised learning, and reinforcement learning.

Supervised learning is a machine learning method that derives a single function from the training data.

Among the functions that may be thus derived, a function that outputs a continuous range of values may be referred to as a regressor, and a function that predicts and outputs the class of an input vector may be referred to as a classifier.

In supervised learning, an artificial neural network can be trained with training data that has been given a label.

Here, the label may refer to a target answer (or a result value) to be guessed by the artificial neural network when the training data is inputted to the artificial neural network.

Throughout the present specification, the target answer (or a result value) to be guessed by the artificial neural network when the training data is inputted may be referred to as a label or labeling data.

Throughout the present specification, assigning one or more labels to training data in order to train an artificial neural network may be referred to as labeling the training data with labeling data.

Training data and labels corresponding to the training data together may form a single training set, and as such, they may be inputted to an artificial neural network as a training set.

Meanwhile, the training data represents a plurality of features, and the labeling the label on the training data can mean that the feature represented by the training data is labeled. In this case, the training data can represent the feature of the input object in the form of a vector.

Using training data and labeling data together, the artificial neural network may derive a correlation function between the training data and the labeling data. Then, through evaluation of the function derived from the artificial neural network, a parameter of the artificial neural network may be determined (optimized).

Unsupervised learning is a machine learning method that learns from training data that has not been given a label.

More specifically, unsupervised learning may be a training scheme that trains an artificial neural network to discover a pattern within given training data and perform classification by using the discovered pattern, rather than by using a correlation between given training data and labels corresponding to the given training data.

Examples of unsupervised learning include, but are not limited to, clustering and independent component analysis.

Examples of artificial neural networks using unsupervised learning include, but are not limited to, a generative adversarial network (GAN) and an autoencoder (AE).

GAN is a machine learning method in which two different artificial intelligences, a generator and a discriminator, improve performance through competing with each other.

The generator may be a model generating new data that generates new data based on true data.

The discriminator may be a model recognizing patterns in data that determines whether inputted data is from the true data or from the new data generated by the generator.

Furthermore, the generator may receive and learn from data that has failed to fool the discriminator, while the discriminator may receive and learn from data that has succeeded in fooling the discriminator. Accordingly, the generator may evolve so as to fool the discriminator as effectively as possible, while the discriminator evolves so as to distinguish, as effectively as possible, between the true data and the data generated by the generator.

An auto-encoder (AE) is a neural network which aims to reconstruct its input as output.

More specifically, AE may include an input layer, at least one hidden layer, and an output layer.

Since the number of nodes in the hidden layer is smaller than the number of nodes in the input layer, the dimensionality of data is reduced, thus leading to data compression or encoding.

Furthermore, the data outputted from the hidden layer may be inputted to the output layer. Given that the number of nodes in the output layer is greater than the number of nodes in the hidden layer, the dimensionality of the data increases, thus leading to data decompression or decoding.

Furthermore, in the AE, the inputted data is represented as hidden layer data as interneuron connection strengths are adjusted through training. The fact that when representing information, the hidden layer is able to reconstruct the inputted data as output by using fewer neurons than the input layer may indicate that the hidden layer has discovered a hidden pattern in the inputted data and is using the discovered hidden pattern to represent the information.

Semi-supervised learning is machine learning method that makes use of both labeled training data and unlabeled training data.

One semi-supervised learning technique involves reasoning the label of unlabeled training data, and then using this reasoned label for learning. This technique may be used advantageously when the cost associated with the labeling process is high.

Reinforcement learning may be based on a theory that given the condition under which a reinforcement learning agent can determine what action to choose at each time instance, the agent can find an optimal path to a solution solely based on experience without reference to data.

The Reinforcement Learning can be mainly performed by a Markov Decision Process (MDP).

Explaining the Markov Decision Process, firstly, the environment in which the agent has the necessary information to do the following actions is given, secondly, it is defined how the agent behaves in the environment, thirdly, i it is defined how to give reward or penalty to the agent, and fourthly, the best policy is obtained by repeatedly experiencing until the future reward reaches its peak.

An artificial neural network is characterized by features of its model, the features including an activation function, a loss function or cost function, a learning algorithm, an optimization algorithm, and so forth. Also, the hyperparameters are set before learning, and model parameters can be set through learning to specify the architecture of the artificial neural network.

For instance, the structure of an artificial neural network may be determined by a number of factors, including the number of hidden layers, the number of hidden nodes included in each hidden layer, input feature vectors, target feature vectors, and so forth.

Hyperparameters may include various parameters which need to be initially set for learning, much like the initial values of model parameters. Also, the model parameters may include various parameters sought to be determined through learning.

For instance, the hyperparameters may include initial values of weights and biases between nodes, mini-batch size, iteration number, learning rate, and so forth. Furthermore, the model parameters may include a weight between nodes, a bias between nodes, and so forth.

Loss function may be used as an index (reference) in determining an optimal model parameter during the learning process of an artificial neural network. Learning in the artificial neural network involves a process of adjusting model parameters so as to reduce the loss function, and the purpose of learning may be to determine the model parameters that minimize the loss function.

Loss functions typically use means squared error (MSE) or cross entropy error (CEE), but the present disclosure is not limited thereto.

Cross-entropy error may be used when a true label is one-hot encoded. One-hot encoding may include an encoding method in which among given neurons, only those corresponding to a target answer are given 1 as a true label value, while those neurons that do not correspond to the target answer are given 0 as a true label value.

In machine learning or deep learning, learning optimization algorithms may be deployed to minimize a cost function, and examples of such learning optimization algorithms include gradient descent (GD), stochastic gradient descent (SGD), momentum, Nesterov accelerate gradient (NAG), Adagrad, AdaDelta, RMSProp, Adam, and Nadam. GD includes a method that adjusts model parameters in a direction that decreases the output of a cost function by using a current slope of the cost function.

The direction in which the model parameters are to be adjusted may be referred to as a step direction, and a size by which the model parameters are to be adjusted may be referred to as a step size.

Here, the step size may mean a learning rate.

GD obtains a slope of the cost function through use of partial differential equations, using each of model parameters, and updates the model parameters by adjusting the model parameters by a learning rate in the direction of the slope.

SGD may include a method that separates the training dataset into mini batches, and by performing gradient descent for each of these mini batches, increases the frequency of gradient descent.

Adagrad, AdaDelta and RMSProp may include methods that increase optimization accuracy in SGD by adjusting the step size, and may also include methods that increase optimization accuracy in SGD by adjusting the momentum and step direction. Adam may include a method that combines momentum and RMSProp and increases optimization accuracy in SGD by adjusting the step size and step direction. Nadam may include a method that combines NAG and RMSProp and increases optimization accuracy by adjusting the step size and step direction.

Learning rate and accuracy of an artificial neural network rely not only on the structure and learning optimization algorithms of the artificial neural network but also on the hyperparameters thereof. Therefore, in order to obtain a good learning model, it is important to choose a proper structure and learning algorithms for the artificial neural network, but also to choose proper hyperparameters.

In general, the artificial neural network is first trained by experimentally setting hyperparameters to various values, and based on the results of training, the hyperparameters can be set to optimal values that provide a stable learning rate and accuracy.

The vehicle controller 1200 may be implemented using at least one among application specific integrated circuits (ASICs), digital signal processors (DSPs), digital signal processing devices (DSPDs), programmable logic devices (PLDs), field [programmable gate arrays (FPGAs), processors, controllers, micro-controllers, microprocessors, and other electronic units for performing other functions.

The vehicle user interface 1300, which serves for communication between a vehicle and a vehicle user, may receive an input signal from the user, may transfer the received input signal to the vehicle controller 1200, and may be controlled by the vehicle controller 1200 to provide the user with information the vehicle has. The vehicle user interface 1300 may include, but is not limited to, an input module, an internal camera, a biometric detection module, and an output module.

The biometric detection module of the vehicle user interface 1300 may include a plurality of electrocardiographic sensors 1310 as illustrated in FIG. 3.

The electrocardiographic sensor 1310 may be mounted on a vehicle seat and obtain the patch signal, and may provide the obtained patch signal to the vehicle controller 1200 or the vehicle communicator 1100.

The electrocardiographic sensor 1310 may be activated in response to starting of a vehicle, and may be activated by the vehicle controller 1200 when the vehicle is started.

The input module is for receiving information from a user. The data collected by the input module may be analyzed by the vehicle controller 1200 and processed by user's control command.

The input module of the vehicle user interface 1300 may receive, from the driver, a signal for requesting a switch from the defensive automatic driving mode to the aggressive automatic driving mode and may provide the received signal to the vehicle controller 1200.

The input module may receive an input of a vehicle destination from the user, and may provide the destination to the vehicle controller 1200.

The input module may input, to the vehicle controller 1200, a signal for designating and deactivating at least one sensor module among a plurality of sensor modules of the object detector 1400 according to an input from the user.

The input module may be disposed inside the vehicle. For example, the input module may be disposed in one area of a steering wheel, one area of an instrument panel, one area of a seat, one area of each pillar, one area of a door, one area of a center console, one area of a head lining, one area of a sun visor, one area of a windshield, or one area of a window.

The output module serves to generate a visual, auditory, or tactile output. The output unit may output a sound or an image.

The output module may include at least one among a display module, a sound output module, and a haptic output module.

The display module may display graphic objects corresponding to various information.

The display module may include at least one of a liquid crystal display (LCD), a thin film transistor liquid crystal display (TFT LCD), an organic light emitting diode (OLED), a flexible display, a 3D display, or an e-ink display.

The display module may form an interactive layer structure with a touch input module, or may be integrally formed with the touch input module to implement a touch screen.

The display module may be implemented as a head up display (HUD). When the display module is implemented as an HUD, the display module may include a project module, and output information through an image projected onto a windshield or a window.

The display module may include a transparent display. The transparent display may be attached to a window shield or a window.

The transparent display may display a predetermined screen with a predetermined transparency. In order to be transparent, the transparent display may include, for example, at least one among a transparent thin film electroluminescent (TFEL), a transparent organic light-emitting diode (OLED), a transparent liquid crystal display (LCD), a transmissive transparent display, and a transparent light emitting diode (LED). The transparency of the transparent display may be adjusted.

The vehicle user interface 1300 may include a plurality of display modules.

The display module may be disposed in one area of the steering wheel, one area of the instrument panel, one area of the seat, one area of each pillar, one area of the door, one area of the center console, one area of the head lining, or one area of the sun visor, or may be implemented on one area of the windshield or one area of the window.

The sound output module may convert an electrical signal provided from the vehicle controller 1200 into an audio signal. The sound output module may include at least one speaker.

The haptic output module may generate a tactile output. For example, the haptic output module may operate to vibrate a steering wheel, a seatbelt, and a seat so that the user may recognize an output.

The object detector 1400, which serves to detect an object positioned outside a vehicle, may generate object information on the basis of sensing data, and may transfer the generated object information to the vehicle controller 1200. Examples of the object may include various objects related to the driving of the vehicle, such as a lane, another vehicle, a pedestrian, a motorcycle, a traffic signal, light, a road, a structure, a speed bump, a landmark, and an animal.

The object detector 1400 may include, as the plurality of sensor modules, a camera module, light imaging detection and ranging (LIDAR), an ultrasonic sensor, radio detection and ranging (RADAR), and an infrared sensor.

The object detector 1400 may generate information about an environment around the vehicle using the plurality of sensor modules.

According to an embodiment, the object detector 1400 may further include other elements in addition to the described elements, or may not include portion of the described elements.

The radar may include an electromagnetic wave transmitting module and an electromagnetic wave receiving module. The radar may be implemented using a pulse radar method or a continuous wave radar method in terms of radio wave emission principle. The radar may be implemented using a frequency modulated continuous wave (FMCW) method or a frequency shift keying (FSK) method according to a signal waveform in a continuous wave radar method.

The radar may detect an object based on a time-of-flight (TOF) method or a phase-shift method via an electromagnetic wave, and detect the location of the detected object, the distance to the detected object, and the relative speed of the detected object.

The radar may be disposed at an appropriate position outside the vehicle for sensing an object disposed at the front, back, or side of the vehicle.

The lidar may include a laser transmitting module, and a laser receiving module. The lidar may be embodied using the time of flight (TOF) method or in the phase-shift method.

The lidar may be implemented using a driving method or a non-driving method.

When the lidar is embodied in the driving method, the lidar may rotate by means of a motor, and detect an object near the vehicle. When the lidar is implemented in the non-driving method, the lidar may detect an object within a predetermined range with respect to the vehicle by means of light steering. The vehicle may include a plurality of non-driving lidars.

The lidar may detect an object using the time of flight (TOF) method or the phase-shift method via laser light, and detect the location of the detected object, the distance from the detected object and the relative speed of the detected object.

The lidar may be disposed at an appropriate position outside the vehicle for sensing an object disposed at the front, back, or side of the vehicle.

An imaging unit may be positioned in an appropriate location outside the vehicle, for example, a front portion, rear portion, right side mirror, or left side mirror of the vehicle. The imaging unit may be a mono camera, but is not limited thereto, and may be a stereo camera, an around view monitoring (AVM) camera, or a 360-degree camera.

The imaging unit may be disposed adjacent to a front wind shield inside the vehicle to obtain an image of an environment in front of the vehicle. Alternatively, the imaging unit may be disposed near a front bumper or radiator grill.

The imaging unit may be disposed adjacent to a rear glass inside the vehicle to obtain an image of an environment in a rear of the vehicle. Alternatively, the imaging unit may be disposed near a rear bumper, a trunk, or a tail gate.

The imaging unit may be disposed adjacent to at least one among side windows inside the vehicle to obtain an image of an environment on a side of the vehicle. Furthermore, the imaging unit may be disposed near a fender or a door.

The ultrasonic sensor may include an ultrasonic transmitting module, and an ultrasonic receiving module. The ultrasonic sensor may detect an object based on ultrasonic waves, and detect the location of the detected object, the distance from the detected object, and the relative speed of the detected object.

The ultrasonic sensor may be disposed at an appropriate position outside the vehicle for sensing an object at the front, back, or side of the vehicle.

The infrared sensor may include an infrared transmitting module, and an infrared receiving module. The infrared sensor may detect an object based on infrared light, and detect the location of the detected object, the distance from the detected object, and the relative speed of the detected object.

The infrared sensor may be disposed at an appropriate position outside the vehicle for sensing an object at the front, back, or side of the vehicle.

The vehicle controller 1200 may control the overall operation of the object detector 1400.

The vehicle controller 1200 may compare data sensed by the radar, the lidar, the ultrasonic sensor, and the infrared sensor with pre-stored data so as to detect or classify an object.

The vehicle controller 1200 may detect an object and perform tracking of the object based on the obtained image. The vehicle controller 1200 may perform the calculation of the distance to the object and the calculation of the speed relative to the object by using an image processing algorithm.

For example, the vehicle controller 1200 may obtain the distance information from the object and the relative speed information of the object from the obtained image based on the change of size of the object over time.

For example, the vehicle controller 1200 may obtain the distance information from the object and the relative speed information of the object through, for example, a pin hole model and road surface profiling.

The vehicle controller 1200 may detect an object and perform tracking of the object based on the reflected electromagnetic wave reflected back from the object. The vehicle controller 1200 may perform operations such as calculation of the distance to the object and calculation of the relative speed of the object based on the electromagnetic waves.

The vehicle controller 1200 may detect an object, and perform tracking of the object based on the reflected laser light reflected back from the object. Based on the laser light, the vehicle controller 1200 may perform operations such as calculation of the distance to the object and calculation of the relative speed of the object based on the laser light.

The vehicle controller 1200 may detect an object and perform tracking of the object based on the reflected ultrasonic wave reflected back from the object. The vehicle controller 1200 may perform operations such as calculation of the distance to the object and calculation of the relative speed of the object based on the reflected ultrasonic wave.

The vehicle controller 1200 may detect an object and perform tracking of the object based on the reflected infrared light reflected back from the object. The vehicle controller 1200 may perform operations such as calculation of the distance to the object and calculation of the relative speed of the object based on the infrared light.

Depending on the embodiment, the object detector 1400 may include a separate processor from the vehicle processor 1200. In addition, the radar, the lidar, the ultrasonic sensor, and the infrared sensor may each include a processor.

When a processor is included in the object detector 1400, the object detector 1400 may be operated under the control of the processor controlled by the vehicle controller 1200.

The driving manipulator 1500 may receive a user input for driving. In the case of a manual mode, the vehicle may be driven on the basis of a signal provided by the driving manipulator 1500.

The vehicle driver 1600 may electrically control the driving of various apparatuses in the vehicle. The vehicle driver 1600 may electrically control operation of a power train, chassis, door/window, safety device, lamp, and air conditioner in the vehicle.

The operator 1700 may control various operations of the vehicle. The operator 1700 may operate in the autonomous driving mode.

The operator 1700 may include a driving module, an unparking module, and a parking module.

Depending on the embodiment, the operator 1700 may further include constituent elements other than the constituent elements to be described, or may not include some of the constitute elements.

The operator 1700 may include a processor under the control of the vehicle controller 1200. Each module of the operator 1700 may include a processor individually.

Depending on the embodiment, when the operator 1700 is implemented as software, it may be a sub-concept of the vehicle controller 1200.

The driving module may perform driving of the vehicle.

The driving module may receive object information from the object detection unit 1400, and provide a control signal to a vehicle driving module to perform the driving of the vehicle.

The driving module may receive a signal from an external device through the vehicle communicator 1100, and provide a control signal to the vehicle driving module, so that the driving of the vehicle 1000 is performed.

The unparking module may perform unparking of the vehicle.

The unparking module may receive navigation information from a navigation module to provide a control signal to a vehicle driving module to perform the unparking of the vehicle.

The unparking module may receive object information from the object detector 1400 to provide a control signal to the vehicle driving module to perform the unparking of the vehicle.

The unparking module may receive a signal from an external device via the vehicle communicator 1100 to provide a control signal to the vehicle driving module to perform the unparking of the vehicle.

The parking module may perform parking of the vehicle.

The parking module may receive navigation information from the navigation module to provide a control signal to the vehicle driving module to perform the parking of the vehicle.

The parking module may receive object information from the object detector 1400 to provide a control signal to the vehicle driving module to perform the parking of the vehicle.

The parking module may receive a signal from an external device via the vehicle communicator 1100 to provide a control signal to the vehicle driving module to perform the parking of the vehicle.

The navigation module may provide navigation information to the vehicle controller 1200. The navigation information may include at least one among map information, set destination information, route information according to destination setting, information on various objects on the route, lane information, and present location information of the vehicle.

The navigation module may provide, to the vehicle controller 1200, a map of a parking lot into which the vehicle has come. When the vehicle has entered the parking lot, the vehicle controller 1200 may receive the parking lot map from the navigation module, and may generate map data by projecting, onto the parking lot map, a calculated movement route and fixed identification information.

The navigation module may include a memory. The memory may store navigation information. The navigation information may be updated by information received through the vehicle communicator 1100. The navigation module may be controlled by an internal processor, or may operate by receiving an external signal, for example, a control signal from the vehicle controller 1200, but the present disclosure is not limited thereto.

The driving module of the operator 1700 may be provided with the navigation information from the navigation module, and may provide a control signal to the vehicle driving module so that driving of the vehicle is performed.

The sensor 1800 may sense the state of the vehicle 1000 using a sensor mounted on the vehicle, that is, a signal related to the state of the vehicle, and obtain movement route information of the vehicle according to the sensed signal. The sensor 1800 may provide the obtained movement route information to the vehicle controller 1200.

The sensor 1800 may include a posture sensor (for example, a yaw sensor, a roll sensor, and a pitch sensor), a collision sensor, a wheel sensor, a speed sensor, a tilt sensor, a weight sensor, a heading sensor, a gyro sensor, a position module, a vehicle forward/reverse movement sensor, a battery sensor, a fuel sensor, a tire sensor, a steering sensor by rotation of a steering wheel, a vehicle interior temperature sensor, a vehicle interior humidity sensor, an ultrasonic sensor, an illuminance sensor, an accelerator pedal position sensor, and a brake pedal position sensor, but is not limited thereto.

The sensor 1800 may acquire sensing signals for information such as vehicle posture information, vehicle collision information, vehicle direction information, vehicle position information (GPS information), vehicle angle information, vehicle speed information, vehicle acceleration information, vehicle tilt information, vehicle forward/reverse movement information, battery information, fuel information, tire information, vehicle lamp information, vehicle interior temperature information, vehicle interior humidity information, a steering wheel rotation angle, vehicle exterior illuminance, pressure on an acceleration pedal, and pressure on a brake pedal.

The sensor 1800 may further include an acceleration pedal sensor, a pressure sensor, an engine speed sensor, an air flow rate sensor (AFS), an air temperature sensor (ATS), a water temperature sensor (WTS), a throttle position sensor (TPS), a TDC sensor, a crank angle sensor (CAS).

The sensor 1800 may generate vehicle status information based on sensing data. The vehicle status information may be information generated based on data sensed by various sensors included in the inside of the vehicle.

The vehicle status information may include at least one among posture information of the vehicle, speed information of the vehicle, tilt information of the vehicle, weight information of the vehicle, direction information of the vehicle, battery information of the vehicle, fuel information of the vehicle, tire air pressure information of the vehicle, steering information of the vehicle, vehicle interior temperature information, vehicle interior humidity information, pedal position information, and vehicle engine temperature information.

The vehicle storage 1900 may be electrically connected to the vehicle controller 1200. The vehicle storage 1900 may store basic data for each part of a lane changing device of an autonomous vehicle, control data for controlling operation of each part of the lane changing device of the autonomous vehicle, and input/output data. The vehicle storage 1900 may be various storage devices such as a ROM, a RAM, an EPROM, a flash drive, and a hard drive, in terms of hardware. The vehicle storage 1900 may store a variety of data for overall operation of a vehicle, in particular, driver tendency information, such as a program or the like for processing or controlling of the vehicle controller 1200. The vehicle storage 1900 may be integrally formed with the vehicle controller 1200, or implemented as a sub-component of the vehicle controller 1200.

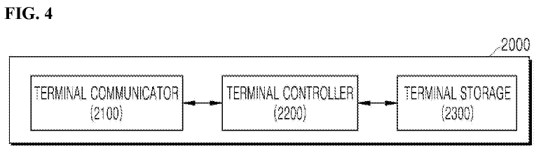

Referring to FIG. 4, the vehicular electrocardiogram measurement device 2000 provided to the user terminal may include a terminal communicator 2100, a terminal controller 2200, and a terminal storage 2300.

According to an embodiment, the vehicular electrocardiogram measurement device may include other elements in addition to the elements illustrated in FIG. 4 and described below, or may not include portion of the elements illustrated in FIG. 4 and described below.

The terminal communicator 2100 may be a module for performing communication with an external device. Here, the external device may be the electrocardiographic sensor 1310 of the vehicle or the server 3000.

The terminal communicator 2100 may include at least one among a transmission antenna, a reception antenna, a radio frequency (RF) circuit capable of implementing various communication protocols, and an RF element in order to perform communication.

The terminal communicator 2100 may support short-range communication using at least one among Bluetooth, RFID, infrared communication, UWB, ZigBee, NFC, Wi-Fi, Wi-Fi Direct, and wireless USB.

The terminal controller 2200 may receive, via the terminal communicator 2100, the patch signal obtained by the electrocardiographic sensor 1310, and may, on the basis of the input patch signal, generate a first request signal for requesting contact of a user's body with the patch of the electrocardiographic sensor 1310 if a first condition is not satisfied, generate a second request signal for requesting to take off clothes if a second condition is not satisfied, generate a third request signal for requesting to stop moving if a third condition is not satisfied, and may determine a stress level on the basis of the patch signal as the first to third conditions are satisfied.

Here, the first condition may be that an absolute value of a voltage of the patch signal exceeds a predetermined first voltage value, the second condition may be that a peak-to-peak voltage value of the patch signal exceeds a predetermined second voltage value, and the third condition may be that the peak-to-peak voltage of the patch signal is measured periodically.

The terminal controller 2200 may determine whether a body of an occupant is in contact with the electrocardiographic sensor 1310 on the basis of a threshold value for differentiating on/off of current.

The terminal controller 2200 may determine that contact is faulty (current off) when the absolute value of the voltage of the wave obtained through the electrocardiographic sensor 1310 remains at a value equal to or smaller than a first voltage value that is a predetermined threshold value for determining on/of, for example, about 0.1 mV, during a certain period of time, for example, about five seconds.

The terminal controller 2200 may determine that contact is established (current on) when there is a period in which the absolute value of the voltage of the wave obtained through the electrocardiographic sensor 1310 exceeds the first voltage value that is a predetermined threshold value for determining on/of, for example, about 0.1 mV, during the certain period of time, for example, about five seconds.

When it is determined that contact is faulty, the terminal controller 2200 may generate the first request signal for inducing contact of a body with the patch of the electrocardiographic sensor 1310, and may provide the generated first request signal via an interface of the user terminal.

The terminal controller 2200 may determine, on the basis of the magnitude of the peak-to-peak voltage of the wave obtained through the electrocardiographic sensor 1310, whether normal signal acquisition of the electrocardiographic sensor 1310 is possible when the occupant wears clothes, i.e., whether it is necessary to take off the clothes to obtain a signal due to the thickness of the clothes of the occupant.

The terminal controller 2200 may determine that it is necessary to take off clothes when the peak-to-peak voltage value of the wave obtained through the electrocardiographic sensor 1310 is equal to or smaller than a second voltage value, for example, about 1.5 mV, that is a predetermined threshold value for determining whether to request to take off clothes.

The vehicle controller 1200 may determine that it is not necessary to take off clothes when the peak-to-peak voltage value of the wave obtained through the electrocardiographic sensor 1310 exceeds a second voltage value, for example, about 1.5 mV, that is a predetermined threshold value for determining whether to request to take off clothes.

When it is determined that taking off clothes is necessary, the terminal controller 2200 may generate the second request signal for inducing the occupant to take off clothes, and may provide the generated second request signal via the interface of the user terminal.

The terminal controller 2200 may determine, on the basis of a period of the peak-to-peak voltage value of the wave obtained through the electrocardiographic sensor 1310, whether normal signal acquisition of the electrocardiographic sensor 1310 for measuring a stress level is possible, i.e., whether it is necessary to stop movement of the occupant.

The terminal controller 2200 may determine that movement of the occupant is required to be stopped when the period at which the peak-to-peak voltage value of the wave obtained through the electrocardiographic sensor 1310 is generated is not constant during a certain period of time, such as about 30 seconds, for example when a 1-sigma interval of a normal distribution curve obtained by measuring and normalizing a generation time interval of the peak-to-peak voltage value exceeds about 30%.

The terminal controller 2200 may refer to a typical period at which the peak-to-peak voltage value of the wave obtained through the electrocardiographic sensor 1310 is generated, so as to determine that movement of the occupant is required to be stopped when the generation time interval of the peak-to-peak voltage value exceeds about two seconds or is equal to or shorter than about 0.3 seconds during a certain period of time, such as about 30 seconds.

The terminal controller 2200 may determine that movement of the occupant is required to be stopped when the number of occurrences of the peak-to-peak voltage value of the wave obtained through the electrocardiographic sensor 1310 is equal to or less than about 30 times or exceeds about 200 times during a certain period of time, such as about 30 seconds.

The terminal controller 2200 may determine that movement of the occupant is not required to be stopped when the period at which the peak-to-peak voltage of the wave obtained through the electrocardiographic sensor 1310 is generated is constant during a certain period of time, such as about 30 seconds, for example when the 1-sigma interval of the normal distribution curve obtained by measuring and normalizing the generation time interval of the peak-to-peak voltage value is about 30% or less.

The terminal controller 2200 may refer to a typical period at which the peak-to-peak voltage value of the wave obtained through the electrocardiographic sensor 1310 is generated, so as to determine that movement of the occupant is not required to be stopped when the generation time interval of the peak-to-peak voltage value exceeds about 0.3 seconds and is equal to or shorter than about two seconds during a certain period of time, such as about 30 seconds.

The terminal controller 2200 may determine that movement of the occupant is not required to be stopped when the number of occurrences of the peak-to-peak voltage value of the wave obtained through the electrocardiographic sensor 1310 exceeds about 30 times and is equal to or smaller than about 200 times during a certain period of time, such as about 30 seconds.

As the first condition, the second condition, and the third condition are satisfied, the terminal controller 2200 may detect a peak value of a QRS wave composite signal through the electrocardiographic sensor 1310, may calculate a heart rate of the occupant on the basis of a time interval between peak values, and may detect an autonomic nervous system balance and a stress index (SI), as described in Table 1, of a person being measured, i.e., may determine a stress level by analyzing the heart rate calculated from the peak values of an electrocardiogram by applying a heart rate variability (HRV) analysis technology.

The terminal controller 2200 may detect whether a seat belt of a vehicle is fastened via the terminal communicator 2100, and may obtain the patch signal from the electrocardiographic sensor 1310 when a predetermined time, for example, about five seconds, has elapsed since the seat belt was fastened, according to a result of the detection. Here, the time between when the seat belt is fastened and when the patch signal is obtained may be determined in consideration of a time required for an automatic vehicle function check and stabilization of the occupant, who is the driver, after the seat belt is fastened.

After initially determining the stress level, the terminal controller 2200 may periodically calculate a heart rate on the basis of the patch signal obtained from the electrocardiographic sensor 1310, and may determine the stress level again on the basis of the patch signal when an absolute value of a difference between the heart rate of a current period and the heart rate of a previous period is at least a predetermined value, for example, about 10 times. That is, the terminal controller 2200 may determine the stress level again to recognize an abnormal state of the occupant when an abnormal change in the heart rate is detected while driving the vehicle.

The terminal storage 2300 is electrically connected to the terminal controller 2200. The terminal storage 2300 may be various storage devices such as a ROM, a RAM, an EPROM, a flash drive, and a hard drive, in terms of hardware. The terminal storage 2300 may be integrally formed with the terminal controller 2200, or implemented as a sub-component of the vehicle controller 2200.

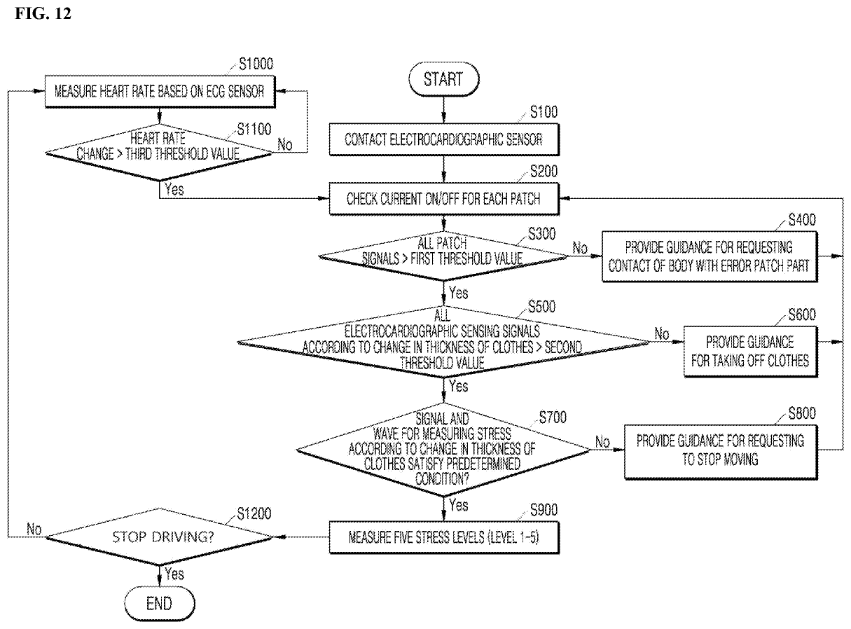

FIG. 12 is an operation flowchart illustrating a vehicular electrocardiogram measurement method according to an embodiment of the present disclosure.

When an occupant of a vehicle sits on a vehicle seat, the occupant may be in contact with the patch of the electrocardiographic sensor 1310 disposed in a back portion of the vehicle seat (S100).

The vehicle controller 1200 or the terminal controller 2200 may obtain the patch signal generated by the electrocardiographic sensor 1310 mounted on the vehicle seat.

In detail, the vehicle controller 1200 or the terminal controller 2200 may check on/off of current for each patch of the plurality of electrocardiographic sensors 1310 after about five seconds have elapsed since a seat belt was fastened (S200).

The vehicle controller 1200 or the terminal controller 2200 may determine whether the body of the occupant is in contact with the electrocardiographic sensor 1310 according to whether the absolute values of voltage magnitudes of all of patch signals obtained through the electrocardiographic sensor 1310 exceed a first threshold value that is a predetermined voltage value for determining on/off during a certain period of time, for example, about five seconds (S300).

When there is a patch which provides a patch signal having a voltage magnitude, the absolute value of which remains equal to or less than the first threshold value during the certain period of time, the vehicle controller 1200 or the terminal controller 2200 may provide, via an interface, a guidance for requesting contact of the patch with the body (S400). Thereafter, the vehicle controller 1200 or the terminal controller 2200 may re-perform an operation of checking on/off of current for each patch of the plurality of electrocardiographic sensors 1310.

When there is no patch which provides a patch signal having a voltage magnitude, the absolute value of which remains equal to or less than the first threshold value during the certain period of time, the vehicle controller 1200 or the terminal controller 2200 may determine whether it is necessary to take off clothes to obtain an electrocardiographic signal due to the thickness of clothes of the occupant according to whether the magnitude of the peak-to-peak voltage value of the wave obtained through the electrocardiographic sensor 1310 exceeds a second threshold value (S500).

Here, the certain period of time for checking on/off of current may be initially set to about five seconds and may be reduced to about one second when the checking is performed again.

When the peak-to-peak voltage value of the wave obtained through the electrocardiographic sensor 1310 is equal to or less than a second threshold value that is a voltage value for determining whether to request to take off clothes, the vehicle controller 1200 or the terminal controller 2200 may provide, via an interface, a guidance for requesting to take off clothes (S600). Thereafter, the vehicle controller 1200 or the terminal controller 2200 may re-perform an operation of checking on/off of current for each patch of the plurality of electrocardiographic sensors 1310.

When the peak-to-peak voltage value of the wave obtained through the electrocardiographic sensor 1310 exceeds the second threshold value that is a voltage value for determining whether to request to take off clothes, the vehicle controller 1200 or the terminal controller 2200 may determine whether movement of the occupant is required to be stopped after a certain period of time, for example, about 10 seconds, has elapsed considering a time for taking off clothes according to predetermined conditions, for example, regularity of the period and the magnitude of the peak-to-peak voltage value of the wave obtained through the electrocardiographic sensor 1310 (S700).

Here, the vehicle controller 1200 or the terminal controller 2200 may determine whether the peak-to-peak voltage value of the wave obtained through the electrocardiographic sensor 1310 exceeds the second threshold value that is a predetermined voltage value for determining whether to request to take off clothes by analyzing signals provided during a time during which the peak-to-peak voltage value may be measured about four to six times, for example, during about five seconds.

When the peak-to-peak voltage value, which exceeds the second threshold value, of the wave obtained through the electrocardiographic sensor 1310 is not generated periodically, the vehicle controller 1200 or the terminal controller 2200 may provide, via an interface, a guidance for requesting to stop movement (S800). Thereafter, the vehicle controller 1200 or the terminal controller 2200 may re-perform an operation of checking on/off of current for each patch of the plurality of electrocardiographic sensors 1310.

When the peak-to-peak voltage value, which exceeds the second threshold value, of the wave obtained through the electrocardiographic sensor 1310 is generated periodically, the vehicle controller 1200 or the terminal controller 2200 may calculate a stress level as described in Table 1 after elapse of a certain period of time, for example, about 30 seconds (S900).

The vehicle controller 1200 or the terminal controller 2200 may determine whether vehicle driving continues after calculating the stress level (S1200).

When vehicle driving is stopped, the vehicle controller 1200 or the terminal controller 2200 may end operation, and when the vehicle is being driven, the vehicle controller 1200 or the terminal controller 2200 may periodically calculate a heart rate on the basis of the patch signal obtained from the electrocardiographic sensor 1310 (S1000), and may determine whether an absolute value of a difference between the heart rate of a current period and the heart rate of a previous period exceeds a predetermined value, for example, about 10 times (S1100).

When the absolute value of the difference between the heart rate of a current period and the heart rate of a previous period is equal to or less than the predetermined value, the vehicle controller 1200 or the terminal controller 2200 may re-perform an operation of periodically calculating the heart rate on the basis of the patch signal obtained from the electrocardiographic sensor 1310.

When the absolute value of the difference between the heart rate of a current period and the heart rate of a previous period exceeds the predetermined value, the vehicle controller 1200 or the terminal controller 2200 may re-perform an operation of checking on/off for each patch of the plurality of electrocardiographic sensors 1310.

According to an embodiment of the present disclosure, an error situation that may occur when measuring an electrocardiogram of a driver or occupant is recognized by using unique characteristics of a wave of an electrocardiographic signal and notifies the error state to the driver or occupant so as to compensate for the error state, and thus the electrocardiogram may be measured accurately.

According to an embodiment of the present disclosure, an electrocardiogram may be prevented from being measured in a situation in which an electrocardiogram measurement error may occur due to movement of a driver or occupant or thick clothes of the driver or occupant, without installing an additional device other than an electrocardiographic sensor disposed in a seat of a vehicle.

The above-mentioned embodiments can be implemented as computer-readable codes in a program-recorded medium. The computer readable medium includes all types of recording devices in which data readable by a computer system readable can be stored. Examples of the computer readable medium include a hard disk drive (HDD), a solid state disk (SSD), a silicon disk drive (SDD), a read-only memory (ROM), a random-access memory (RAM), CD-ROM, a magnetic tape, a floppy disk, an optical data storage device, and the like, and it may also be implemented in the form of a carrier wave (for example, transmission over the Internet). In addition, the computer may include a processor or a controller. Therefore, the above description should not be construed as limiting and should be considered illustrative. The scope of the present disclosure should be determined by rational interpretation of the appended claims, and all changes within the scope of equivalents of the present disclosure are included in the scope of the present disclosure.

What is claimed is:

1. A vehicular electrocardiogram measurement device comprising:
an electrocardiographic sensor which is installed in a seat of a vehicle and obtains a patch signal; and
a controller configured to, on the basis of the patch signal obtained by the electrocardiographic sensor, generate a first request signal for requesting contact of a user's body with a patch of the electrocardiographic sensor if a first condition is not satisfied, generate a second request signal for requesting to take off clothes if a second condition is not satisfied, generate a third request signal for requesting to stop moving if a third condition is not satisfied, and determine a stress level on the basis of the patch signal as the first condition, the second condition, and the third condition are satisfied,
wherein the first condition is that an absolute value of a voltage of the patch signal exceeds a predetermined first voltage value,
the second condition is that a peak-to-peak voltage value of the patch signal exceeds a predetermined second voltage value, and
the third condition is that the peak-to-peak voltage value of the patch signal is measured periodically.

2. The vehicular electrocardiogram measurement device of claim 1,
wherein the electrocardiographic sensor is activated in response to starting of the vehicle, and
the controller obtains the patch signal when a predetermined time has elapsed since a seat belt of the seat of the vehicle was fastened.

3. The vehicular electrocardiogram measurement device of claim 1, wherein the controller periodically calculates a heart rate on the basis of the patch signal, and determines the stress level on the basis of the patch signal when an absolute value of a difference between the heart rate of a current period and the heart rate of a previous period is at least a predetermined value.

4. The vehicular electrocardiogram measurement device of claim 1, further comprising:
a communicator,
wherein the communicator receives compensation data for setting the first voltage value and the second voltage value on the basis of a downlink grant of a 5G network connected to drive in an automatic driving mode.

5. The vehicular electrocardiogram measurement device of claim 4,
wherein the automatic driving mode comprises a defensive automatic driving mode and an aggressive automatic driving mode,
the controller generates a control signal for requesting a switch from the defensive automatic driving mode to the aggressive automatic driving mode when the stress level determined during driving in the defensive automatic driving mode is at least a predetermined reference level, and
the communicator transmits the control signal on the basis of an uplink grant of the 5G network connected to drive in the automatic driving mode.

6. The vehicular electrocardiogram measurement device of claim 4, further comprising:
a user interface,
wherein the automatic driving mode comprises a defensive automatic driving mode and an aggressive automatic driving mode,
the controller generates a control signal for requesting a switch from the defensive automatic driving mode to the aggressive automatic driving mode when the stress level determined during driving in the defensive automatic driving mode is at least a predetermined reference level and a signal for requesting the switch from the defensive automatic driving mode to the aggressive automatic driving mode is received via the user interface, and
the communicator transmits the control signal on the basis of an uplink grant of the 5G network connected to drive in the automatic driving mode.

7. A vehicular electrocardiogram measurement device connected to a vehicle comprising an electrocardiographic sensor which is installed in a seat and is activated in response to starting of the vehicle and obtains a patch signal, the vehicular electrocardiographic measurement device comprising:
a controller configured to, on the basis of the patch signal obtained by the electrocardiographic sensor, generate a first request signal for requesting contact of a user's body with a patch of the electrocardiographic sensor if a first condition is not satisfied, generate a second request signal for requesting to take off clothes if a second condition is not satisfied, generate a third request signal for requesting to stop moving if a third condition is not satisfied, and determine a stress level on the basis of the patch signal as the first condition, the second condition, and the third condition are satisfied,
wherein the first condition is that an absolute value of a voltage of the patch signal is less than a predetermined first voltage value,
the second condition is that a peak-to-peak voltage value of the patch signal is at least a predetermined second voltage value, and
the third condition is that the peak-to-peak voltage value of the patch signal is measured periodically.

8. The vehicular electrocardiogram measurement device of claim 7, wherein the controller obtains the patch signal when elapse of a predetermined time is recognized after a seat belt of the seat of the vehicle is fastened.

9. The vehicular electrocardiogram measurement device of claim 7, wherein the controller periodically calculates a heart rate on the basis of the patch signal, and determines the stress level on the basis of the patch signal when an absolute value of a difference between the heart rate of a current period and the heart rate of a previous period is at least a predetermined value.

10. A vehicular electrocardiogram measurement method comprising:
obtaining a patch signal through an electrocardiographic sensor installed in a seat of a vehicle; and
on the basis of the patch signal obtained by the electrocardiographic sensor, generating a first request signal for requesting contact of a user's body with a patch of the electrocardiographic sensor if a first condition is not satisfied, generating a second request signal for requesting to take off clothes if a second condition is not satisfied, generating a third request signal for requesting to stop moving if a third condition is not satisfied, and determining a stress level on the basis of the patch signal as the first condition, the second condition, and the third condition are satisfied,
wherein the first condition is that an absolute value of a voltage of the patch signal exceeds a predetermined first voltage value,
the second condition is that a peak-to-peak voltage value of the patch signal exceeds a predetermined second voltage value, and
the third condition is that the peak-to-peak voltage value of the patch signal is measured periodically.

11. The vehicular electrocardiogram measurement method of claim 10, further comprising:
activating the electrocardiographic sensor in response to starting of the vehicle,
wherein the obtaining the patch signal comprises obtaining the patch signal through the electrocardiographic sensor when a predetermined time has elapsed since a seat belt of the seat of the vehicle was fastened.

12. The vehicular electrocardiogram measurement method of claim 10, further comprising periodically calculating a heart rate on the basis of the patch signal, and determining the stress level on the basis of the patch signal when an absolute value of a difference between the heart rate of a current period and the heart rate of a previous period is at least a predetermined value.

13. The vehicular electrocardiogram measurement method of claim 10, further comprising receiving compensation data for setting the first voltage value and the second voltage value on the basis of a downlink grant of a 5G network connected to drive in an automatic driving mode.

14. The vehicular electrocardiogram measurement method of claim 13, further comprising:
generating a control signal for requesting a switch from a defensive automatic driving mode to an aggressive automatic driving mode when the stress level determined during driving in the defensive automatic driving mode is at least a predetermined reference level; and
transmitting the control signal on the basis of an uplink grant of the 5G network connected to drive in the automatic driving mode,
wherein the automatic driving mode comprises the defensive automatic driving mode and the aggressive automatic driving mode.

15. The vehicular electrocardiogram measurement method of claim 13, further comprising:
generating a control signal for requesting a switch from a defensive automatic driving mode to an aggressive automatic driving mode when the stress level determined during driving in the defensive automatic driving mode is at least a predetermined reference level and a signal for requesting the switch from the defensive automatic driving mode to the aggressive automatic driving mode is received; and
transmitting the control signal on the basis of an uplink grant of the 5G network connected to drive in the automatic driving mode,
wherein the automatic driving mode comprises the defensive automatic driving mode and the aggressive automatic driving mode.

16. A computer-readable recording medium in which a vehicular electrocardiogram measurement program is recorded, the program causing a computer to perform:
obtaining a patch signal through an electrocardiographic sensor installed in a seat of a vehicle; and
on the basis of the patch signal, generating a first request signal for requesting contact of a user's body with a patch of the electrocardiographic sensor if a first condition is not satisfied, generating a second request signal for requesting to take off clothes if a second condition is not satisfied, generating a third request signal for requesting to stop moving if a third condition is not satisfied, and determining a stress level on the basis of the patch signal as the first condition, the second condition, and the third condition are satisfied,
wherein the first condition is that an absolute value of a voltage of the patch signal exceeds a predetermined first voltage value,
the second condition is that a peak-to-peak voltage value of the patch signal exceeds a predetermined second voltage value, and
the third condition is that the peak-to-peak voltage value of the patch signal is measured periodically.
